(19) United States
(12) Patent Application Publication
(10) Pub. No.: US 2025/0204695 A1
(43) Pub. Date: Jun. 26, 2025

(54) A PIECE OF FURNITURE OR A TEXTILE ELEMENT

(71) Applicant: ELEDA S.R.L., Milano (IT)

(72) Inventor: Renato CAIMI, Milano (IT)

(21) Appl. No.: 18/849,883

(22) PCT Filed: Mar. 24, 2023
(86) PCT No.: PCT/IB2023/052923

§ 371 Date: Sep. 23, 2024

(30) Foreign Application Priority Data
Mar. 24, 2022 (IT) 102022000005879

(51) Int. Cl. A47C 31/00; A47C 7/18

(52) U.S. Cl. CPC A47C 31/004; A47C 7/18

(57) ABSTRACT

A piece of furniture or a textile element comprising a layer of sound-absorbing material and a layer of a material for shielding against magnetic and electromagnetic fields is described. The textile element may be a curtain comprising a first layer of sound-absorbing fabric and a second layer of EMI shielding material, wherein the first layer and the second layer are configured to be at least partially in contact with each other.

### FIELD OF APPLICATION

The present invention relates in general to the sector of furniture intended for homes, working environments or recreational facilities of any kind.

More particularly, the present invention relates to the sector of furniture having sound absorption and electromagnetic interference protection characteristics.

### PRIOR ART

It is known that, when a sound wave emitted in a closed environment encounters a surface, part of its energy passes through the surface, part is absorbed by the impact with the surface and part is reflected back into the environment.

If, in an environment, the reflective surface area is high, the environment may be acoustically very disturbed since the sound waves produced inside it are amplified with an effect similar to that of an echo.

In order to improve the acoustics in an environment, without structural modifications, it is known to envisage the use of sound-absorbing materials in the environment, in particular next to the surfaces which delimit the said environment (for example vertical walls and/or ceiling). Sound-absorbing materials, as is known, have the property that they absorb at least a part of the acoustic energy and reduce the amount of reflected energy.

WO 2013/113800, in the name of the Applicant, describes a sound-absorbing panel comprising a padding layer comprising heat-bonded synthetic fibres in which said padding layer has a first thickness. At least one portion of said panel has a variable density, which is greater in the region of its outer layer and smaller in the region of its inner layer.

EP 3227504 describes an element and a sound-absorbing system.

### SUMMARY OF THE INVENTION

For the purposes of the present description and the attached claims, the term “furniture” is to be understood as comprising: a sofa, a settee, a bench, an armchair, an easy chair, a chair, a pouf, a stool, a bed or any other piece of furniture that allows a person to sit and/or lie down, a table, a coffee table, a shelf, a bookcase (or part thereof) or any other piece of furniture which has a support surface for supporting objects. The term “furniture” also includes an element for separating an environment, such as a screen, a partition or a dividing wall. The term “furniture” also includes a design element to be applied to a wall and/or to be installed near a wall in order to improve the acoustics of an environment.

For the purposes of this description and the attached claims, the term “textile element” will include a cushion, a mattress, a curtain, a sofa cover, an armchair cover, a rug, a duvet cover or the like.

The inventor has defined the aim of improving the well-being of persons when they are in a closed environment, such as their home, or in a working environment or in a leisure facility (for example a discotheque, a cinema, a theatre or a conference room).

The inventor has noted that the well-being of a person depends on many factors, including the exposure to noise and the exposure to electromagnetic waves, also due to the presence of electrical and electronic equipment in the aforementioned environments.

The inventor has considered that the aforementioned aims relating to well-being may be achieved with a piece of furniture or a textile element comprising a layer of sound-absorbing material and a layer of material for shielding against magnetic and electromagnetic fields.

Preferably, the layer of sound-absorbing material comprises (or is essentially composed of) a fabric or a non-woven fabric.

Preferably, the layer of shielding material comprises (or is essentially composed of) a fabric or a non-woven fabric.

The layer of sound-absorbing material may be made of a natural or artificial fabric with certain specific resistance properties Rs and mass porosity properties MP, as will be discussed in detail here below. The inventor has advantageously discovered that fibrous materials with a specific resistance (which will also be indicated, more simply, as Rs) less than 414 Pa s/m or greater than 2368 Pa s/m and a mass porosity (which will also be indicated, more simply, as MP) less than 60% or greater than 81% have a poor performance in terms of sound absorbance; that fibrous materials with a specific resistance Rs of between 527 Pa s/m and 1552 Pa s/m and a mass porosity MP of between 66% and 79% have a good performance in terms of sound-absorbance; and that fibrous materials with a specific resistance Rs of between 723 Pa s/m and 1213 and a mass porosity MP of between 74% and 77% have an optimum performance in terms of sound absorbance.

The definition of these properties advantageously allows the identification of materials suitable for the sound-absorbing layer according to the present invention.

According to the invention, the layer of shielding material comprises a fabric for protection against electromagnetic interference. For example, the fabric may be a woven fabric lined with an electrically conductive material such as metal, for example silver or silver alloy.

The fabric may consist of natural and/or synthetic fibres. For example, it may comprise (or be essentially composed of) polyamide fibres.

The thickness of the layer of metallic material may be about 20 μm or about 30 μm or in a range between about 20 μm and about 40 μm or in a range between about 22 μm and about 30 μm.

According to a first aspect, the present invention provides a piece of furniture or a textile element comprising a layer of sound-absorbing material and a layer of an EMI shielding material for shielding against magnetic and electromagnetic fields.

The piece of furniture may comprise a substantially rigid structure and open-cell padding layer. The padding layer may be sound-absorbing.

According to embodiments, the layer of sound-absorbing material is the outermost layer.

The layer of sound-absorbing material may comprise a fabric or a padding layer.

The layer of EMI shielding material for shielding against magnetic and electromagnetic fields may be in direct contact with the layer of sound-absorbing material, or with an interposed layer, or may be arranged between the interposed layer and a padding layer. or may be arranged between the layer of sound-absorbing material and the padding material, or inside the structure, or between the padding layer and the structure.

The structure may comprise wood or the like, metal or a composite material.

The layer of EMI shielding material may comprise a fabric at least partially covered with an electrically conductive material.

The electrically conductive fabric may comprise natural or artificial fibres and is at least covered with a metal, for example silver or silver alloy.

The layer of sound-absorbing material may comprise a fibrous material with the following properties:
specific resistance of between 527 and 1552 [Pa s/m]; and
mass porosity of between 66% and 79%.

According to embodiments, the layer of sound-absorbing material has a specific resistance of between 723 and 1213 [Pa s/m] and mass porosity of between 74% and 77%.

The layer of sound-absorbing material preferably comprises a fabric, preferably an artificial fabric.

According to one aspect of the invention, the textile element comprises a curtain comprising a first layer of sound-absorbing fabric and a second layer of EMI shielding material, wherein the first layer and the second layer are configured to be at least partially in contact with each other.

A distance, or an air gap, is provided between the first layer and the second layer of the curtain in order to increase the sound-absorbing performance. The air gap may be substantially constant, but may also not be constant and have a sinusoidal type progression.

The curtain may also comprise a third layer for shielding the second layer.

The third layer of the curtain may comprise a sound-absorbing fabric. A distance, or an air gap, may be provided between the second layer and the third layer in order to increase the sound-absorbing performance. The air gap may be substantially constant, but may also not be constant and have a sinusoidal type progression.

The electrically conductive fabric of the curtain may comprise a layer of natural or artificial fibres and may be at least partially covered with a metal, for example silver or silver alloy.

### BRIEF DESCRIPTION OF THE DRAWINGS

The present invention will become completely clear from the following detailed description, provided by way of a non-limiting example, to be read with reference to the attached drawings in which:

FIG. 1 shows in schematic form a piece of furniture (sofa) according to a first embodiment of the present invention;

FIG. 2 shows in schematic form a piece of furniture (sofa) according to a second embodiment of the present invention;

FIG. 3 shows in schematic form a piece of furniture (wall panel) according to a third embodiment of the present invention;

FIG. 3A shows in schematic form a piece of furniture (wall panel) according to a fourth embodiment of the present invention;

FIG. 4 shows in schematic form a textile element according to an embodiment of the present invention;

FIG. 5 shows in schematic form a cross-section through the EMI shielding fabric;

FIG. 6A shows an exploded view of a curtain according to a first embodiment of the present invention; and

FIG. 6B shows an exploded view of a curtain according to a second embodiment.

### DETAILED DESCRIPTION

For the purposes of the present description and the claims, it must be understood that, except where otherwise indicated, all the numbers which express values, quantities, percentages and the like must be interpreted as being preceded, in each case, by the term “about”. Furthermore, all the ranges include every combination of the end values of the range indicated and include all the sub-ranges, which may also not be specifically indicated in the present description.

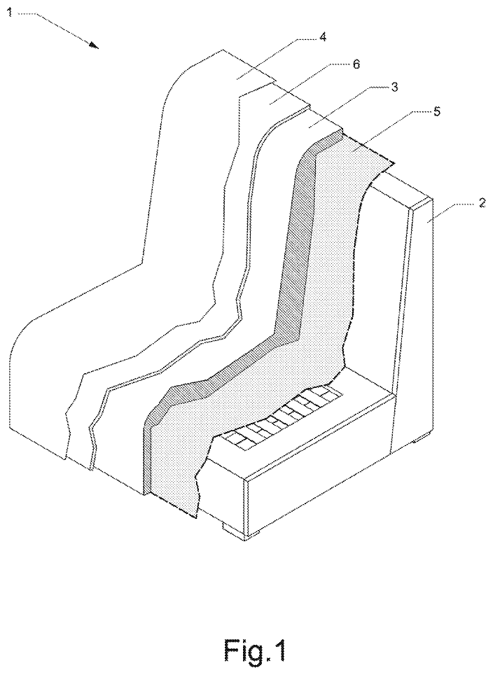

FIG. 1 shows a cross-sectional view of a sofa 1 according to an embodiment of the present invention.

The sofa 1 comprises a substantially rigid structure 2 which may be made of wood, or metal or a thermoplastic material. The sofa 1 may also comprise an open-cell padding layer 3 which may be sound-absorbing, an outer fabric layer 4 which may be sound-absorbing and a layer 5 of EMI shielding material, namely for shielding against electromagnetic interference (from magnetic and electromagnetic fields).

According to embodiments, a further interposed layer 6 of fabric, which may be sound-absorbing or non sound-absorbing, is also provided.

The structure 1 may be made of wood-like or wood-derived material (for example plywood, chipboard, medium density fibreboard-MDF) or metal, where necessary provided with belts. The structure 1 may also be made of a plastic material, a composite material or any combination of wood, metal, plastic and composite material.

Alternatively, the structure 2 may be replaced by a plastic material in the form of small spheres or the like (“bag” type sofa).

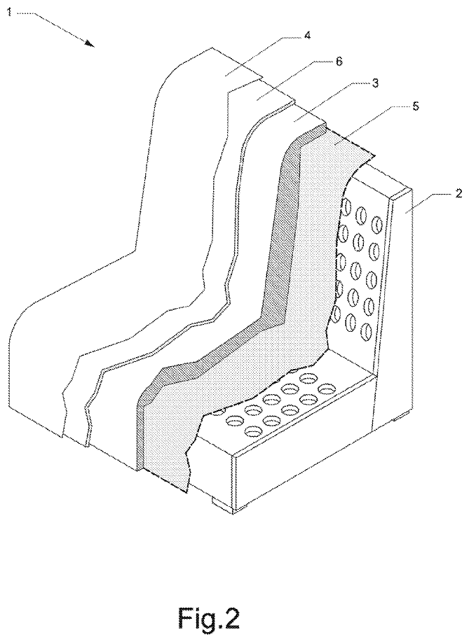

As shown in the embodiment of FIG. 2, one or more panels of the structure 2 advantageously may have holes or passages for the air and therefore also the noise in order to exploit better the characteristics of the sound-absorbing materials.

The padding layer 3 may have a single density or, according to embodiments, a plurality of different densities, for example so as to obtain different degrees of softness between the seat and backrest in order to ensure comfort.

The padding layer 3 may comprise open cells so as to allow the passage of air which, together with the sound-absorbing fabric 4, creates the ideal conditions for absorbing the sound and noise.

An interposed layer 6, characterized by a low air flow resistance, may be present between the padding layer 3 and the outer (sound-absorbing) fabric layer 4, in order to improve the aesthetic appearance of the sofa.

The sofa described above, advantageously, allows the person sat on the sofa to be protected, at least partially, from the electromagnetic waves, reducing exposure thereto.

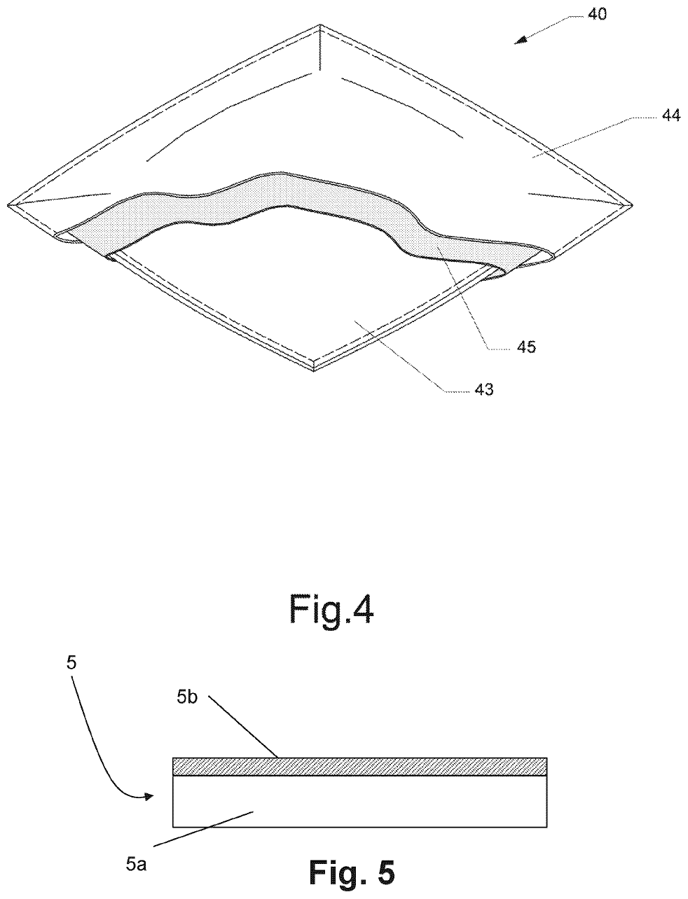

With reference to FIG. 5, the layer of shielding material 5 comprises a fabric or a non-woven fabric 5a at least partially covered with a layer of electrically conductive material 5b, for example metallic material. The metal of the metallic layer 5b may comprise silver or silver alloy.

The fabric may consist of natural and/or synthetic fibres. For example, it may comprise (or be essentially composed of) polyamide fibres.

The thickness of the layer of metallic material may be about 20 μm or about 30 μm or in a range between 20 μm and about 40 μm or in a range between about 22 μm and about 30 μm .

The layer 5 of EMI shielding material may be positioned equally well between one of the following layers:
between the layer 4 and the interposed layer 6;
between the interposed layer 6 and the padding layer 3;
between the sound-absorbing fabric layer 4 and the padding layer 3;
between any one of the above layers (4, 6, 3) and the structure 2;
between the bottom covers (similar to the front covers) and the structure 2; and
inside the structure 2.

For armchairs without a structure, the EMI shielding layer may be advantageously positioned on the side where a person is intended to sit.

In another embodiment, the outer fabric layer 4 could also not have sound-absorbing functions. In this case, the padding layer 3 must have sound-absorbing functions.

In a further embodiment, the outer fabric layer 4 and the padding layer 3 may be both made of sound-absorbing material.

The concepts described above may be easily applied also, for example, to separators, furniture units, wall panels and bed headboards and may also be applied to cushions or quilts which obviously will not have the supporting structure, but which may house the EMI shielding material interposed between the covering fabric 4 and any one of the inner padding layers, or fabrics containing the open-cell padding, or other materials, for example natural or synthetic downs.

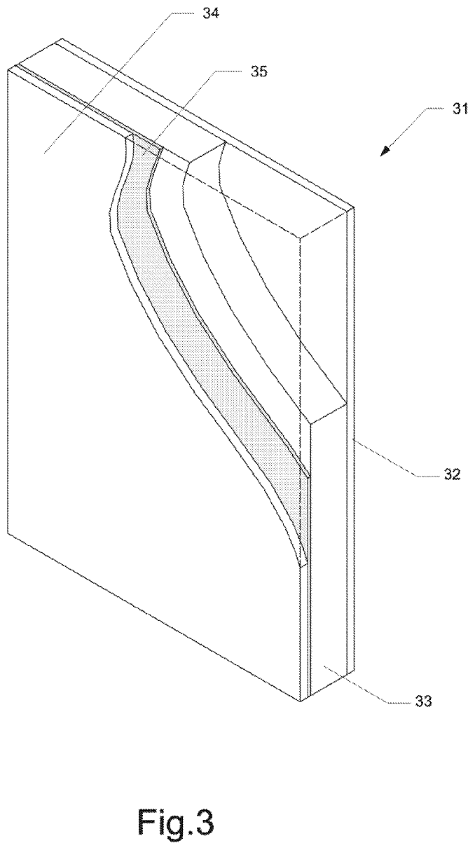

FIG. 3 shows in schematic form, by way of example, a wall panel 31 comprising a support structure 32 (which is optional), an open-cell padding layer 33, an outer fabric layer 34, which is typically sound-absorbing, and a layer 35 of EMI shielding material, namely for shielding against electromagnetic interference (from magnetic or electromagnetic fields). The layer 35 of EMI shielding material may be arranged in a position different from that shown in FIG. 3. The description of the layer 35 will not be repeated since said layer is typically like the layer 5 described above.

The structure 32 may be made of wood-like or wood-derived material (for example plywood, chipboard, medium density fibreboard-MDF) or metal. The structure 32 may also be made of a plastic material, a composite material or any combination of wood, metal, plastic and composite material.

FIG. 3A shows in schematic form, by way of example, another embodiment of a panel 31 configured to be arranged close to a wall. The panel 31 comprises a support structure 32 (which is optional), an open-cell padding layer 33, an outer fabric layer 34, which is typically sound-absorbing, and a layer 35 of EMI shielding material, namely for shielding against electromagnetic interference (from magnetic or electromagnetic fields). The layer 35 of EMI shielding material may be arranged in a position different from that shown in FIG. 3. The description of the layer 35 will not be repeated since said layer is typically like the layer 5 described above.

Advantageously, according to this embodiment, the layer 35 extends over at least part of at least one edge of the panel 31. In this way, in a composition of several adjacent panels, the layer 35 of EMI shielding material along the edge of a first panel will be in electrical contact with the layer 35 of EMI shielding material along the edge of a second panel, adjacent to the first panel.

The structure 32 may be made of wood-like or wood-derived material (for example plywood, chipboard, medium density fibreboard-MDF) or metal. The structure 32 may also be made of a plastic material, a composite material or any combination of wood, metal, plastic and composite material.

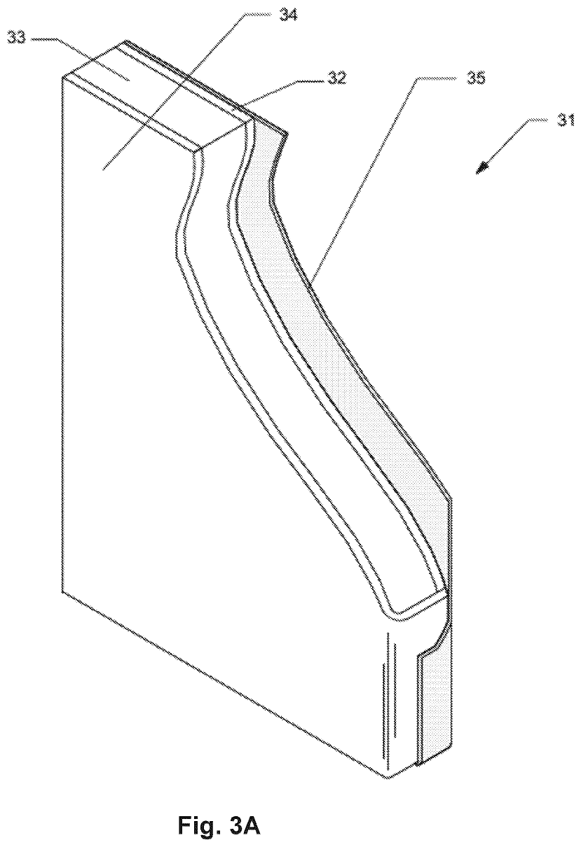

FIG. 4 shows a textile element, for example a cushion 40. The cushion 40 comprises an open-cell padding layer 43, an outer fabric layer 44, which is typically sound-absorbing, and a layer 45 of EMI shielding material, namely for shielding against electromagnetic interference (from magnetic or electromagnetic fields).

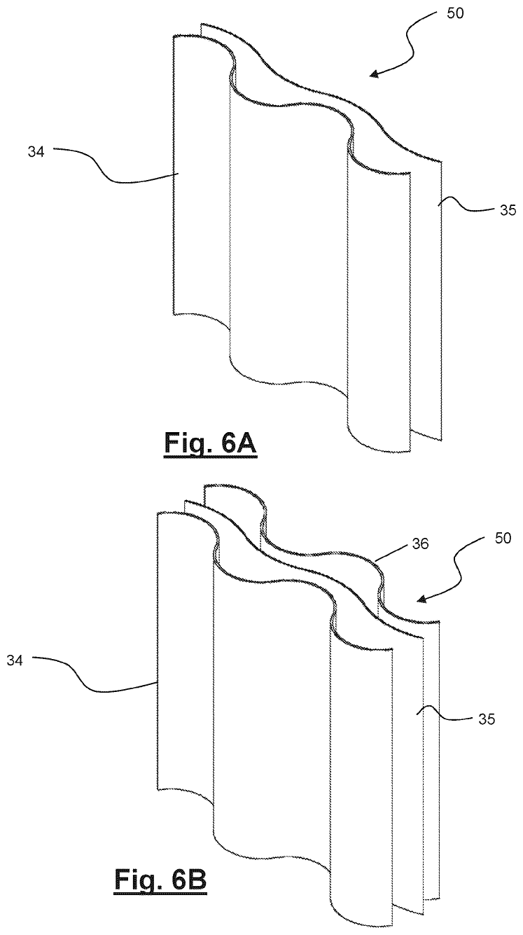

FIGS. 6A and 6B show in schematic form two different embodiments of a curtain. The two figures show the various layers as separate from each other, in a kind of exploded view. During use, the curtain may have the layers which are fastened to each other, at least along separate points, lines or surfaces.

In a first embodiment (FIG. 6A), the curtain 50 comprises a first outer layer 34 of sound-absorbing fabric and a layer 35 of EMI shielding material. The first outer layer 34 of sound-absorbing fabric is configured to be arranged facing the environment (typically a room) to be screened acoustically and protected from magnetic and electromagnetic fields. The layer 35 of EMI shielding material will instead be directed towards the wall or the window in the environment.

Advantageously, a distance, or an air gap, is provided between the layer 34 and the layer 35 in order to increase the sound-absorbing performance. The layers of sound-absorbing fabric and EMI shielding material are as described above for the other embodiments.

In a second embodiment (FIG. 6B), the curtain comprises a first outer layer 34 of sound-absorbing fabric and a layer 35 of EMI shielding material. The first outer layer 34 of sound-absorbing fabric is configured to be arranged facing the environment (typically a room) to be screened acoustically and protected magnetic and electromagnetic fields. The layer 35 of EMI shielding material will instead be directed towards the wall or the window in the environment.

According to the second embodiment, the curtain also comprises a third layer 36 covering the second layer 35 of EMI shielding material. The layer 36 may be advantageously a layer of sound-absorbing fabric or a layer of fabric which is not sound-absorbing or only partially sound-absorbing. One function of the third layer is to increase the sound-absorbing characteristics of the curtain. Another function is to hide the second layer from sight. This aspect is particularly advantageous in the case where the curtain is installed in an environment where a particular confidentiality is required. The third layer 36 allows, in fact, screening of the curtain against external laser microphones, thus providing an improved anti-intrusion curtain.

Advantageously, a distance, or an air gap, is provided between the layer 34 and the layer 35, but also between the layer 35 and the layer 36, in order to increase the sound-absorbing performance. The layers of sound-absorbing fabric and EMI shielding material are as described above for the other embodiments.

1. A piece of furniture or a textile element comprising a layer of a sound-absorbing material and a layer of an electromagnetic interference EMI, shielding material for shielding against magnetic and electromagnetic fields.

2. The piece of furniture according to claim 1, comprising a substantially rigid structure and a padding layer.

3. The piece of furniture according to claim 2, wherein the layer of sound-absorbing material comprises an outermost layer.

4. The piece of furniture according to claim 3, wherein the layer of sound-absorbing material comprises a fabric or a padding layer.

5. The piece of furniture according to claim 2, wherein the layer of an EMI shielding material for shielding against magnetic and electromagnetic fields is in direct contact with the layer of sound-absorbing material or with an interposed layer, or is arranged between the interposed layer and a padding layer, or is arranged between the layer of sound-absorbing material and the padding layer, or inside the structure, or between the padding layer and the structure

6. The piece of furniture or the textile element according to claim 1, wherein the layer of EMI shielding material comprises a fabric at least partially covered with an electrically conductive material.

7. The piece of furniture or the textile element according to claim 6, wherein the electrically conductive fabric comprises a layer of natural or artificial fibres and is at least partially covered with a metal.

8. The piece of furniture or the textile element according to claim 1, wherein the layer of sound-absorbing material comprises a fibrous material having the following properties:
specific resistance of between 527 and 1552 [Pa s/m]; and
mass porosity of between 66% and 79%.

9. The piece of furniture or the textile element according to claim 8, wherein the layer of sound-absorbing material has a specific resistance of between 723 and 1213 [Pa s/m] and mass porosity of between 74% and 77%.

10. The piece of furniture according to claim 2, wherein the padding layer is sound-absorbing.

11. The piece of furniture or the textile element according to claim 8, wherein the layer of sound-absorbing material comprises a fabric, preferably an artificial fabric.

12. The textile element according to claim 1, wherein the textile element comprises a curtain, comprising a first layer of sound-absorbing fabric and a second layer of EMI shielding material, wherein the first layer and the second layer are configured to be at least partially in contact with each other.

13. The textile element according to claim 12, wherein a distance, or an air gap, is provided between the first layer and the second layer in order to increase the sound-absorbing performance.

14. The textile element according to claim 12, also comprising a third layer for shielding the second layer.

15. The textile element according to claim 14, wherein the third layer comprises a sound-absorbing fabric and/or wherein a distance, or an air gap, is provided between the second layer and the third layer, in order to increase the sound-absorbing performance.

16. The textile element according to claim 12, wherein the electrically conductive fabric comprises a layer of natural or artificial fibres and is at least covered with a metal.
